(19) United States
(12) Patent Application Publication
(10) Pub. No.: US 2022/0168201 A1
(43) Pub. Date: Jun. 2, 2022

(54) USE OF DIANHYDROHEXITOL FOR PRESERVING COSMETIC PREPARATIONS

(71) Applicant: ROQUETTE FRERES, Lestrem (FR)

(72) Inventors: Léon MENTINK, Lille (FR); Daniel WILS, Morbecque (FR)

(21) Appl. No.: 17/593,732

(22) PCT Filed: Mar. 27, 2020
(86) PCT No.: PCT/EP2020/058680

§ 371 Date: Sep. 23, 2021

(30) Foreign Application Priority Data
Mar. 28, 2019 (FR) FR1903292

(51) Int. Cl. A61K 8/49; A61Q 19/00

(52) U.S. Cl. CPC A61K 8/4973; A61K 2800/70; A61K 2800/524; A61Q 19/00

(57) ABSTRACT

The present application pertains to the field of cosmetics, more specifically the field of antimicrobial and/or bacteriostatic and/or bactericidal and/or antifungal cosmetic agents, preferably acting on bacterial and/or fungal strains in cosmetic preparations. Proposed is a use of dianhydrohexitol, preferably isosorbide, to reduce or eliminate the effects of the degradation of said preparation contaminated by microorganisms of the human cutaneous microbiome.

### TECHNICAL FIELD

The present application pertains to the field of cosmetics, more specifically the field of antimicrobial and/or bacteriostatic and/or bactericidal and/or antifungal preserving agents, preferably acting on bacterial and/or fungal strains in cosmetic or dermatological preparations. Preferentially, proposed is a use of isosorbide to reduce or eliminate the effects of the degradation of said preparation following contamination by microorganisms of the human cutaneous microbiome or present in the atmosphere. Isosorbide is used alone or additionally with other preserving agents already used for this purpose.

### BACKGROUND ART

In U.S. Pat. No. 9,295,626 by Pilz et al., a method for preserving cosmetic, dermatological and pharmaceutical preparations by using bactericidal compounds based on isosorbide monoester and/or diester is disclosed. This patent presents a minimum inhibitory concentration for isosorbide equal to 10% for many strains (col. 27, Table 2), including in particular the following strains that can be found in the cutaneous microbiome: Aspergillus brasiliensis, Candida albicans, and Staphylococcus aureus.

U.S. Pat. No. 999,300 by Stoer et al. also discloses the use of isosorbide derivatives, herein ethers, as a bactericidal agent for preserving cosmetic compositions, in particular by inhibiting the growth of a multitude of bacterial strains, including Propionobacterium acnes.

### Technical Problem

Human skin is colonized by resident bacterial flora, which constitutes the cutaneous microbiome. Although this flora is non-pathogenic most of the time, in certain abnormal situations it may become pathogenic. A first abnormal situation is that of contamination of the cutaneous microbiome by a pathogenic external microorganism, through contact with a contaminated environment. A second abnormal situation is that of the appearance of an imbalance in the interactions between the microorganisms that constitute the cutaneous microbiome, which results in the proliferation of one microorganism to the detriment of the other microorganisms. Such a proliferation can be generalized over a large area of the skin, or can be more localized, for example in the areas of the skin that are richer in water or warmer, or for example on the face or scalp due to the presence of nutritional reserves for said microorganisms, such as the triglycerides located in the glands at the base of the hair or the hairs. Apart from the potentially harmful consequences for the health of the skin or of the individual, these abnormal situations also cause disorders of a cosmetic nature, in particular visual, tactile, or olfactory, which can adversely affect the individual's comfort or their image, and thus disrupt or degrade their social life.

A cosmetic or dermatological preparation is generally packaged aseptically in an airtight container having a hole through which said product is expelled, usually by mechanical action, in order to be applied to the human body. This expulsion operation systematically brings the cosmetic or dermatological product into contact with the atmosphere or the human body, and in particular with the topical human microbiome, mainly the cutaneous microbiome. This contacting can induce a contamination of said cosmetic or dermatological preparation by microorganisms of a very varied nature: bacteria, yeasts, fungi. These microorganisms can find nutritional organic substances and water in the cosmetic or dermatological preparation, and thus develop there in an uncontrolled manner. Such a proliferation of microorganisms should be avoided because these microorganisms are potentially pathogenic, and can to a lesser extent due to their development, modify the cosmetic or dermatological preparation such that it becomes inactive or its use becomes unpleasant, in particular because of the appearance of bad odors or a change in texture or sensory profile.

There is currently an increased need for consumers to have products of natural origin. The use according to the invention addresses this need, proposing the use of at least dianhydrohexitol, since dianhydrohexitols are molecules of natural origin, produced from cereal-based starch, for example.

### DESCRIPTION OF EMBODIMENTS

It is to the applicant's credit to have discovered, unexpectedly, that non-derived dianhydrohexitols, preferably isosorbide, have antimicrobial, and/or bactericidal and/or bacteriostatic, and/or antifungal effects on microbial strains that cause contamination and physicochemical degradation of cosmetic or dermatological preparations.

The invention more particularly draws on the use of at least one dianhydrohexitol in a cosmetic or dermatological preparation for topical use to limit or prevent the proliferation of microorganisms of the human cutaneous microbiome.

Also proposed is a use of isosorbide as a preserving agent for cosmetic or dermatological preparations, alone or in addition to another known preserving agent.

Use of Dianhydrohexitol in the Preservation of Cosmetic Preparations

The present invention relates to the use of at least one dianhydrohexitol in a cosmetic or dermatological preparation for topical use to limit or prevent the proliferation of microorganisms of the human cutaneous microbiome or those present in the atmosphere.

Preferentially, the microorganisms are selected from bacteria and fungi, and are preferably the cause of the inactivation of cosmetic agents, and/or of the appearance of bad odors, and/or of change in texture of a cosmetic or dermatological preparation.

According to a preferred embodiment, the dianhydrohexitol is selected from isosorbide, isomannide, isoidide, and preferentially isosorbide.

Additionally, the use in a cosmetic or dermatological preparation is made at a dianhydrohexitol content of 0.1% to 50% by weight, preferentially 0.5% to 25% by weight, more preferentially 1% to 25% by weight, even more preferentially from 2% to 15% by weight, and most preferentially 5% to 9% by weight, relative to the total weight of the composition.

According to one embodiment of the use according to the invention, dianhydrohexitol is an antimicrobial and/or bactericidal and/or bacteriostatic and/or antifungal agent, and makes it possible to reduce or prevent the development of bacterial and/or fungal strains coming from the human topical microbiome, in said cosmetic or dermatological preparation.

Preferentially, the bacterial strain is selected from the Propionobacterium spp family, preferentially the bacterial strain is Propionobacterium acnes; and/or the bacterial strain is chosen from the Corynebacterium spp family, preferentially the bacterial strain Corynebacterium xerosis; and/or the bacterial strain is selected from the Staphylococcus spp family, preferentially the bacterial strain is Staphylococcus epidermis; and the fungal strain is selected from the Malassezia spp family, preferentially the fungal strain is Malassezia furfur.

According to a variant of the use according to the invention, dianhydrohexitol is used in combination with other biocides commonly used in cosmetic or dermatological preparations, such as acids, alkali salts of benzoic acid or of sorbic acid or of propionic acid; or of other biocides such as phenoxyethanol or chlorphenesin.

Dianhydrohexitol

The aqueous solution in question can contain only one dianhydrohexitol, or can also contain several of them. These dianhydrohexitols (1,4-3,6-dianhydrohexitols) are isosorbide (1,4-3,6-dianhydrosorbitol), isomannide (1,4-3,6-dianhydromannitol), isoidide (1,4-3,6-dianhydroiditol) and the mixtures of at least two of these products. Preferably, the aqueous solution only contains only one dianhydrohexitol which is isosorbide.

In this regard, the applicant indicates that generally, the dianhydrohexitols are synthesized in the presence of water (or water is generated during their synthesis): the recovery of said dianhydrohexitol in this reaction medium immediately provides a composition in the form of an aqueous solution of dianhydrohexitol which can be used according to the invention. Dianhydrohexitol solutions can in particular be obtained according to the methods described in above-mentioned patent applications EP1287000 and WO03/043959. The choice can be made to keep all or part of the water used during the preparation of the dianhydrohexitol or to eliminate all the water so as to obtain a product in solid form that will be made back into an aqueous solution by simply adding water, which is another possibility for preparing an aqueous solution of dianhydrohexitol that can be used according to the invention.

The applicant specifies that the term “1,4-3,6-dianhydrohexitol” does not include derivatives of 1,4-3,6-dianhydrohexitol, in particular such as 1,4-3,6-dianhydrohexitol ethers or esters.

Cosmetic or Dermatological Preparation

By “cosmetic or dermatological preparation”, the applicant means any composition intended for being placed in contact with human or animal skin.

The cosmetic or dermatological preparation according to the invention comprises, as an active preserving agent, in particular antibacterial and/or antifungal, at least one dianhydrohexitol, preferably selected from isosorbide, isomannide, isoidide, preferentially is isosorbide.

According to a preferred embodiment, the cosmetic or dermatological preparation according to the invention comprises 0.1% to 50% by weight of dianhydrohexitol, preferentially 0.5 to 25%, more preferentially 1% to 25%, even more preferentially 2% to 15%, and most preferentially 5% to 9%.

According to a highly preferential embodiment, the cosmetic or dermatological preparation contains as sole antimicrobial and/or bactericidal and/or bacteriostatic and/or antifungal agent, at least one dianhydrohexitol, preferentially selected from isosorbide, isomannide, and isoidide, preferentially is isosorbide.

The cosmetic or dermatological preparation according to the invention makes it possible to reduce or eliminate the inactivation of cosmetic agents due to microbial degradation, and/or to reduce or eliminate the appearance of bad odors, or the change in texture of said cosmetic or dermatological preparation.

The cosmetic preparation can be a skin product, a hair product, make-up or a hygiene product.

Among the skincare products, the cosmetic preparation according to the invention can be selected from day creams, sun creams, after-sun creams, self-tanners, masks. Among the hair care products, the cosmetic preparation according to the invention is preferentially selected from shampoos, conditioners (creams, masks, lotions), styling products (sprays, gels, waxes), coloring products. Among the make-up products, the cosmetic preparation according to the invention is preferentially selected from foundations, eye shadows. Among the hygiene products, the cosmetic preparation according to the invention is preferably selected from washing gels, shower gels, cleansing or make-up removing wipes, hydroalcoholic solutions or gels, soaps, deodorants, antiperspirants, and body sprays.

### Example

In-Vitro Measurements of the Effect of Isosorbide on Microbial Strains

The samples are prepared under sterile conditions and deposited in microplates (2-ml wells) with the concentrations hereunder. Two analysis controls were carried out:
a positive control corresponding to 0.5% Phenonip®;
a negative control consisting of 0.85% physiological saline solution.

Samples and controls are contaminated with three bacterial strains, namely Staphylococcus epidermidis, Corynebacterium xerosis, and Propionobacterium acnes, at a rate of approximately 1.43×105 to 4.15×105 colony-forming units per milliliter, denoted cfu/ml; and a fungal strain, namely Malassezia furfur, at a rate of approximately 1.50×104 to 3.80×104 cfu/ml. After contamination, the samples are carefully mixed by suction and discharge cycles in order to ensure a uniform distribution of the microorganism. The whole is incubated at 22° C. during 28 days.

The microbial population is sampled and counted at 24 hours and 7, 14, 21 and 28 days for each microbial strain. The contaminated samples are collected and then deposited in serial dilutions in microtiter plates, in the presence of culture medium, which is a saline solution with 0.85% sodium chloride, and a dehydrogenase activity indicator reagent, which is 2,3,5-triphenyltetrazolium chloride (denoted TTC). The results are presented in the following pages with the variation of the microbial population over the 28 days of study, for the tested microbial strain.

The measurements of microbial populations in the samples collected at each time, are carried out according to the following microtiter method, for one sample collection. In the 96 wells with a volume of 250 μL of a microtiter plate, 20 μL of the collected sample are diluted by a factor of 10 by dispersion in 180 μL of Letheen broth (Difco, ref. 268110) containing 1.5% of Tween 80 (Sigma, ref. P1754) and TTC (Sigma, ref. T8877). The microplate is incubated for 48 hours at 32.5° C., and the growth of the microorganisms is monitored by the color change, from colorless to red/pink. The highest reciprocal dilution indicating a growth makes it possible to determine the log number of each microorganism at each time.

The measurements of microbial populations taken at each collection time are expressed as colony forming units/ml.

TABLE 1
Bactericidal effect of isosorbide on the strain
Propionobacterium acnes

| | | Number of colony-forming strains after: | | | | |
|---|---|---|---|---|---|---|
| Dose | Inoculum | 24 h | 7 days | 14 days | 21 days | 28 days |
| Positive | 3.98 × 10⁵ | 4.00 × | 7.00 × | 4.00 × | 1.00 × | 1.00 × |
| control | | 10³ | 10⁴ | 10⁴ | 10⁴ | 10⁴ |
| Negative | 3.98 × 10⁵ | 0 | 0 | 0 | 0 | 0 |
| control | | | | | | |
| 1% | 3.98 × 10⁵ | 0 | 0 | 0 | 0 | 0 |
| 5% | 3.98 × 10⁵ | 0 | 0 | 0 | 0 | 0 |
| 9% | 3.98 × 10⁵ | 0 | 0 | 0 | 0 | 0 |

After 24 hours, no colony-forming strain is observed: isosorbide made it possible to kill the entire inoculum. Isosorbide is a bactericide of the strain Propionobacterium acnes strain.

TABLE 2
Antifungal effect of isosorbide on the Malassezia furfur strain

| Isosorbide | | Number of colony-forming strains after: | | | | |
|---|---|---|---|---|---|---|
| dose | Inoculum | 24 h | 7 days | 14 days | 21 days | 28 days |
| Positive | 1.5 × 10⁴ | 4.00 × | 1.00 × | 7.00 × | 1.00 × | 1.00 × |
| control | | 10³ | 10³ | 10² | 10³ | 10³ |
| Negative | 1.5 × 10⁴ | 0 | 0 | 0 | 0 | 0 |
| control | | | | | | |
| 1% | 1.5 × 10⁴ | 0 | 0 | 0 | 0 | 0 |
| 5% | 1.5 × 10⁴ | 0 | 0 | 0 | 0 | 0 |
| 9% | 1.5 × 10⁴ | 0 | 0 | 0 | 0 | 0 |

After 24 hours, no colony-forming strain is observed: isosorbide made it possible to kill the entire inoculum. Isosorbide is a bactericide of the Malassezia furfur strain.

TABLE 3
Bactericidal effect of isosorbide on the
Corynobacterium xerosis strain

| | | Number of colony-forming strains after: | | | | |
|---|---|---|---|---|---|---|
| Dose | Inoculum | 24 h | 7 days | 14 days | 21 days | 28 days |
| Positive | 3.2 × 10⁵ | 7.00 × | 1.00 × | 7.00 × | 4.00 × | 7.00 × |
| control | | 10⁴ | 10⁴ | 10³ | 10³ | 10³ |
| Negative | 3.2 × 10⁵ | 0 | 0 | 0 | 0 | 0 |
| control | | | | | | |
| 1% | 3.2 × 10⁵ | 4 × 10² | 0 | 0 | 0 | 0 |
| 5% | 3.2 × 10⁵ | 1 × 10² | 0 | 0 | 0 | 0 |
| 9% | 3.2 × 10⁵ | 3.7 × | 0 | 0 | 0 | 0 |
| | | 10² | | | | |

After 24 hours, the number of colony-forming strains is reduced by at least 3 log 10 for the three doses tested. After 7 days, the are no more colony-forming strains: isosorbide is a moderate bactericide of the Corynobacterium xerosis strain.

TABLE 4
Bactericidal effect of isosorbide on the
Staphylococcus epidermis strain

| | | Number of colony-forming strains after: | | | | |
|---|---|---|---|---|---|---|
| Dose | Inoculum | 24 h | 7 days | 14 days | 21 days | 28 days |
| Positive | 1.43 × 10⁵ | 7.00 × | 4.00 × | 7.00 × | 1.00 × | 1.00 × |
| control | | 10⁴ | 10⁴ | 10⁴ | 10⁵ | 10⁵ |
| Negative | 1.43 × 10⁵ | 0 | 0 | 0 | 0 | 0 |
| control | | | | | | |
| 1% | 1.43 × 10⁵ | 4 × 10³ | 0 | 0 | 0 | 0 |
| 5% | 1.43 × 10⁵ | 1 × 10³ | 0 | 0 | 0 | 0 |
| 9% | 1.43 × 10⁵ | 7 × 10² | 0 | 0 | 0 | 0 |

After 24 hours, the number of colony-forming strains is reduced by at least 2 log 10 for the three doses tested. After 7 days, the are no more colony-forming strains: isosorbide is a weak bactericide of the Staphylococcus epidermis strain.

The invention is not limited to the examples described above, given only by way of example, but it encompasses all the alternatives that a person skilled in the art could contemplate in the context of the sought protection.

1. A use of at least one dianhydrohexitol to limit or prevent the proliferation of microorganisms of the human cutaneous microbiome in a cosmetic or dermatological preparation for topical use.

2. The use according to claim 1, wherein the microorganisms are selected from bacteria and fungi, and are preferably the cause of the inactivation of cosmetic agents, and/or of the appearance of bad odors, and/or of change in texture of a cosmetic or dermatological preparation.

3. The use according to claim 1, wherein the dianhydrohexitol is selected from isosorbide, isomannide, isoidide, preferentially isosorbide.

4. The use according to claim 1, in a cosmetic or dermatological preparation with a dianhydrohexitol content of 0.1% to 50% by weight, preferentially from 0.5% to 25% by weight, more preferentially from 1% to 25% by weight, even more preferentially from 2% to 15% by weight, and most preferentially from 5% to 9% by weight, relative to the total weight of the composition.

5. The use according to claim 1, wherein said at least one dianhydrohexitol is a bacteriostatic or/and bactericidal and/or antifungal agent, and that it makes it possible to reduce or prevent the development of bacterial and/or fungal strains coming from the human topical microbiome, in said cosmetic or dermatological preparation.

6. The use according to claim 2, wherein the bacterial strain is selected from the Propionobacterium spp family, preferentially the bacterial strain is Propionobacterium acnes; and/or the bacterial strain is selected from the Corynebacterium spp family, preferentially the bacterial strain Corynebacterium xerosis; and/or the bacterial strain is selected from the Staphylococcus spp family, preferentially the bacterial strain is Staphylococcus epidermis; and in that the fungal strain is selected from the Malassezia spp family, preferentially the fungal strain is Malassezia furfur.

7. The use according to claim 3, wherein the bacterial strain is selected from the Propionobacterium spp family, preferentially the bacterial strain is Propionobacterium acnes; and/or the bacterial strain is selected from the Corynebacterium spp family, preferentially the bacterial strain Corynebacterium xerosis; and/or the bacterial strain is selected from the Staphylococcus spp family, preferentially the bacterial strain is Staphylococcus epidermis; and in that the fungal strain is selected from the Malassezia spp family, preferentially the fungal strain is Malassezia furfur.

8. The use according to claim 4, wherein the bacterial strain is selected from the Propionobacterium spp family, preferentially the bacterial strain is Propionobacterium acnes; and/or the bacterial strain is selected from the Corynebacterium spp family, preferentially the bacterial strain Corynebacterium xerosis; and/or the bacterial strain is selected from the Staphylococcus spp family, preferentially the bacterial strain is Staphylococcus epidermis; and in that the fungal strain is selected from the Malassezia spp family, preferentially the fungal strain is Malassezia furfur.

9. The use according to claim 5, wherein the bacterial strain is selected from the Propionobacterium spp family, preferentially the bacterial strain is Propionobacterium acnes; and/or the bacterial strain is selected from the Corynebacterium spp family, preferentially the bacterial strain Corynebacterium xerosis; and/or the bacterial strain is selected from the Staphylococcus spp family, preferentially the bacterial strain is Staphylococcus epidermis; and in that the fungal strain is selected from the Malassezia spp family, preferentially the fungal strain is Malassezia furfur.
